(19) United States
(12) Patent Application Publication
(10) Pub. No.: US 2023/0168203 A9
(43) Pub. Date: Jun. 1, 2023

(54) Pyrogallarene Chelating and Sequestering Agents

(71) Applicant: Kinsey Spiel, Lindon, UT (US)

(72) Inventor: Paul Spiel, Lindon, UT (US)

(73) Assignee: Kinsey Spiel, Lindon, UT (US)

(21) Appl. No.: 16/553,882

(22) Filed: Aug. 28, 2019

Related U.S. Application Data
(60) Provisional application No. 62/724,516, filed on Aug. 29, 2018.

(51) Int. Cl. G01N 21/78; C07C 13/70

(52) U.S. Cl. CPC G01N 21/78; C07C 13/70

(57) ABSTRACT

Pyrogallarene chelating and sequestering agents are disclosed. The chelating and sequestering agents may be used in a homogeneous environment or heterogeneous environment. In addition, chromogenic properties may be utilized to assess the chelation or sequestration of certain atoms.

### CROSS REFERENCE TO RELATED APPLICATIONS

This application claims priority to U.S. Provisional Patent Application 62/724,516 filed 29 Aug. 2018, which is incorporated by reference in the present application.

### BACKGROUND OF THE INVENTION

### 1. The Field of the Invention

The invention relates to sequestration and chelation. In accordance with various embodiments it relates to the chelation and sequestration of atoms. In addition to chelation, chromogenic properties may be utilized to visualize the chelation of certain atoms or ions.

### 2. The Relevant Technology

One of the most common chelating agents is ethylenediaminetetraacetic acid (EDTA). EDTA is a chelating agent that has a high affinity for metals and is specifically added to solutions to form metal-EDTA complexes. EDTA is a substituted diamine and is applied predominantly in aqueous solutions. EDTA, like a hand, grasps metal ions via its two amines and four terminal carboxylates. It sequesters ions, lowering or eliminating their activity in solution. EDTA was patent in Germany in 1935 by F. Munz. It is commonly used in many industries, as metal ions can cause deleterious effects in several industrial processes and the formulation of many products. It is also widely used in household and food products, commonly to trap metal ions that could catalyze the decomposition of the product. In shampoos, soaps, and laundry detergents EDTA is used to reduce the “hardness” of the water so that other ingredients may better cleanse.

Chelating agents may be bidentate, tridentate, polydentate, etc. EDTA is a type of polydentate or hexadentate ligand that has six donor atoms which it can use to bond to a central atom or ion. EDTA forms very stable complexes, mostly with transition metals but also with main-group ions. Chelating compounds tend to displace monodentates in coordination complexes in solution because of the entropy-favoring effects of displacing multiple monodentates and forming one metal-chelation complex.

In addition to EDTA many sequestering agents have been employed to limit or eliminate the activity of ions in solution. Sequestration agents are often known as chelating agents. Chelation often refers to a type of bonding whereas sequestration refers to the chemical reaction that involves that bonding. For example, a type of sequestering agent could be a crown ether, where the molecule is a large ring containing a number of ether linkages.

### BRIEF SUMMARY OF THE INVENTION

It is an object of the invention to provide a low-cost chelator and sequestering agent having advantages over previous chelators and sequestering agents. It is also an object of the invention to provide a chelator that operates heterogeneously or homogeneously in an aqueous environment.

A method for using a pyrogallarene as a chelating agent may comprise dispensing the pyrogallarene into an aqueous solution. In at least one embodiment the pyrogallarene is a pyrogallol[4]arene. In accordance with various embodiments the pyrogallol[4]arene may be C-propyl pyrogallol[4]arene, C-methyl pyrogallol[4]arene, or C-ethyl pyrogallol[4]arene.

The pyrogallol[4]arene may be used as a homogeneous part of the aqueous solution or as a heterogeneous part of the aqueous solution. A pyrogallol[4]arene or pyroallol[4]arene solution reaction may be observed to see a shift in absorbance color, which may be used to indicate the occurrence of chelation or sequestration. The pyrogallol[4]arene may be mixed in the aqueous solution. The pyrogallol[4]arene may be dispensed into the aqueous solution after being dissolved in an organic solvent. The organic solvent may be one of methanol, ethanol, propanol, butanol, acetone, DMSO, or DMF. Where the aqueous solution is deionized and neutral the pyrogallol[4]arene may be suspended as a colorless colloid, provided the pyrogallol[4]arene is concentrated in the organic solvent so as to produce an aqueous colloid.

In at least one embodiment a method for using a pyrogallarene as a homogeneous chelator comprises dissolving the pyrogallarane in an organic solvent and dispensing the dissolved pyrogallarene into an aqueous solution. The pyrogallarene in the resulting aqueous solution may have a concentration of about 3×10⁻³ M or greater.

In at least one embodiment a method for using a pyrogallarene as a heterogeneous chelator comprises dispensing the pyrogallarene into an aqueous solution containing metal ions, resulting in reduced activity of metal ions in solution or precipitation of chelated metal ions out of solution. The method further comprises mixing the aqueous solution or centrifuging the aqueous solution. The pyrogallarane may be used heterogeneously in a solid chelator, aqueous ion reaction.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific chemical structures and embodiments thereof which are illustrated in the appended drawings. It is appreciated that these drawings are not to be considered limiting in the inventions scope. The invention will be described and explained with additional specificity and detail through the use of the accompanying drawings in which:

FIG. 1 illustrates the chemical structure of a pyrogallarene.

FIG. 2 illustrates the chemical structure of a pyrogallol[4]arene.

FIG. 3 illustrates the chemical structure of a functionalized pyrogallarene.

### DETAILED DESCRIPTION OF THE PREFERRED EMBODIMENTS

The description that follows describes, illustrates, and exemplifies one or more particular embodiments of the present invention in accordance with its principles. This description is not provided to limit the invention to the embodiment or embodiments described herein, but rather to explain and teach the principles of the invention in such a way to enable one of ordinary skill in the art to understand these principles and, with that understanding, be able to apply them to practice not only the embodiment or embodiments described herein, but also other embodiments that may come to mind in accordance with these principles.

The scope of the present invention is intended to cover all such embodiments that may fall within the scope of the appended claims, either literally or under the doctrine of equivalents.

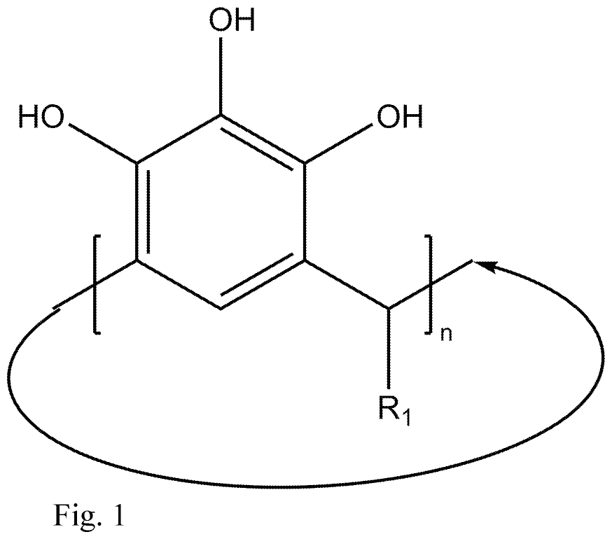

Referring to FIG. 1, a pyrogallarene is illustrated. Pyrogallols are linked together in a ring, n designating the number of pyrogallols linked. A pendant group R₁ operates to link the pyrogallol units. Where R₁ is a propyl group (—CH₂CH₂CH₃) and n equals four the molecule would be C-propyl pyrogallol[4]arene. Similarly, where R₁ is an ethyl group (—CH₂CH₃) and n equals four the molecule would be C-ethyl pyrogallol[4]arene. Where R₁ is a saturated carbon chain (such as an unbranched carbon chain —CₙH₂ₙ₊₁) and n equals four the molecule would be C-alkyl pyrogallol[4]arene. However, as discussed below, R₁ may take a variety of forms.

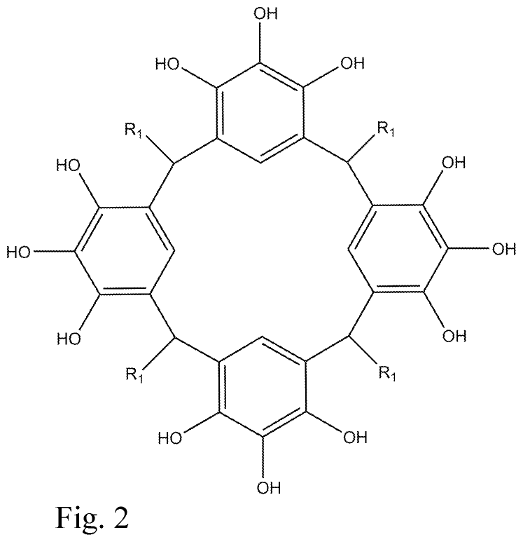

Referring to FIG. 2, a pyrogallol[4]arene is illustrated. Pyrogallol[4]arenes are commonly synthesized through acid-catalyzed condensation reactions of 1:1 molar ratios of pyrogallol and an aldehyde. Each aldehyde adjoins two pyrogallols by way of the acid catalyzed condensation reaction. Upon completion of the condensation reaction the former aldehyde covalently links two pyrogallol molecules and is depicted as R₁ throughout the figures. R₁ is located in the meso position of the aromatic ring. Although R groups are commonly represented as organic chains, R₁ may contain any group, polar or nonpolar. R₁ may also be organic or inorganic. In at least one embodiment R₁ is a hydrocarbon chain. R₁ may be a methyl group (—CH₃), an ethyl group (—CH₂CH₃), or a propyl group (—C₃H₇). R₁ may be branched or unbranched, such as a branched hydrocarbon chain. In accordance with various embodiments R₁ may contain an alcohol. R₁ may contain a sulfur group, sulfoxide, or a sulfonate. R₁ may contain a carboxylic acid. R₁ may contain an amine, a halogen, a ketone, a nitrile, or an ester.

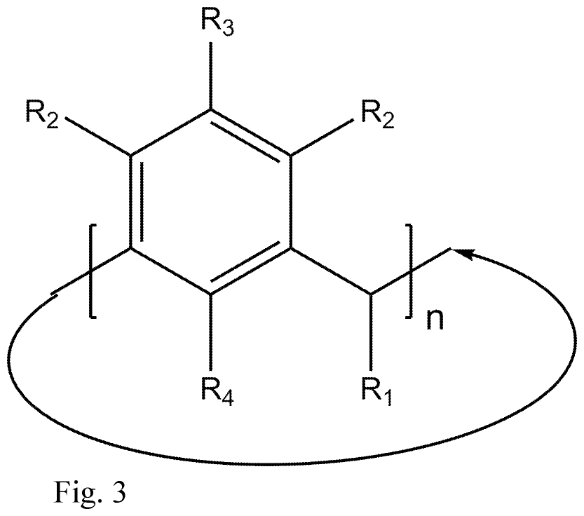

Referring to FIG. 3, a functionalized calixarene is illustrated. R₄ operates as a pendant group on the lower rim of the aromatic ring. Although R groups are commonly represented as organic chains, R₄ may be any pendant group, polar or nonpolar. R₄ may also be composed of organic or inorganic atoms. R₂ and R₃ are pendant groups on the upper rim of the aromatic rings. In at least one embodiment R₂ and R₃ are OH or H groups. In accordance with various embodiments R₂ is a hydroxyl group and R₃ is a hydrogen. The ring size is designated by n. If R₂ and R₃ are hydroxyl groups and R₄ is a hydrogen, making the aromatic ring derived from pyrogallol, four pyrogallols linked together would be a pyrogallol[4]arene, and n would be equal to 4. By implication, if six pyrogallols are linked together the structure would be a pyrogallol[6]arene, and n would be equal to 6.

In at least one embodiment the pyrogallarene operates as a heterogeneous chelator and sequestering agent. “Homogenous” often refers to two reactants in the same state, such as liquid/liquid, gas/gas, or solid/solid, and “heterogeneous” often refers to two reactants in different physical states, such as solid to liquid, liquid to gas, or solid to gas. By way of example, a heterogeneous reaction may occur where an aqueous ion reacts in the presence of a solid catalyst. To illustrate further, C-propyl pyrogallarene is only slightly soluble, generally considered insoluble, in water. Molecules are generally considered insoluble if their concentration in an aqueous solution is less than 0.0001M or if their Ksp is much less than 1. Since, for example, C-propyl pyrogallarene may be considered insoluble in water, it may act as a heterogeneous chelator, sequestering aqueous ions in its solid state. However, it is generally appreciated that a sodium salt or other ionic salt may be made from C-alkyl pyrogallol[4]arenes or other pyrogallarenes to increase their solubility. Insoluble solid pyrogallarene chelation complexes, because of their specific gravity, will fall to the bottom of aqueous solutions and can be filtered out with the chelated ions. Alternatively, the purified aqueous solution supernatant may be siphoned off with the leftover precipitated pyrogallarene chelation complex remaining at the bottom of the solution. Aqueous pyrogallarene chelation complexes may be similarly precipitated out of solution or separated from the supernatant.

In at least one embodiment the pyrogallarene acts as a homogeneous chelator, for example sequestering ions in an aqueous state. In the homogeneous form the pyrogallarene may be dispensed into solution through the means of an additional solvent such as methanol, ethanol, propanol, butanol, acetone, DMSO, DMF, 1,4-dioxane, or other common organic solvents. For example, in at least one embodiment C-ethyl pyrogallol[4]arene may be dissolved in the solvent of acetone, having a concentration of about 7.53×10⁻³ M (moles/liter). Subsequently 1 mL of this solution may be dispensed into a 20 mL aqueous solution, resulting in a dilution and a C-ethyl pyrogallol[4]arene concentration to about 3.77×10⁻³ M. Such as discussed herein, the combination of the 1 mL and 20 mL portions may create a colloid. However, this disclosure is not limited to these specific volumes. Depending upon the ion concentration, pH, and ionic environment the solution may turn a variety of colors, which may be used as an indication of one or more chelation complexes, as illustrated in subsequent examples. Where the pH of the solution is above 8.5 the solution may be pink. In neutral solutions that are deionized, “soft water” solutions, or solutions having approximately 50 ppm or mg/L of total dissolved solids (TDS) the colloid remains white. In fairly hard (210-320 ppm or mg/L) or hard (320+ ppm or mg/L) water that is neutral the colloid will dispense and solid precipitate will form. In at least one embodiment the precipitate is blue, gray, or purple. Because of the specific gravity of the formed precipitate it falls to the bottom of the solution, but in at least one embodiment the precipitate is centrifuged to the bottom of the solution. Depending on the pyrogallarene and solvent used, the precipitate may dissolve back into solution over the period of a week or more. As a result, one embodiment encompasses the separation of the pyrogallarene precipitate from the aqueous mixture prior to precipitate dissolution. Thus, pyrogallarenes may be employed to chelate and sequester ions at least on a parts-per-million (PPM) basis, lowering the activity of ions in solution or removing ions from solution by a chelating precipitate. As well, pyrogallarenes may be used to visually assess the hardness of water.

In various other embodiments the concentration of C-ethyl pyrogallol[4]arene dissolved in acetone is about 3.0×10⁻² M, 1.5×10⁻² M, 5.6×10⁻³ M, or 4.56×10⁻³ M. In testing total dissolved solids in fairly hard water the C-ethyl pyrogallol[4]arene concentration in acetone may exceed about 3.76×10⁻³ M. Where the C-ethyl pyrogallol[4]arene molarity in acetone is too low under the circumstances the subsequent aqueous solution will be pink, chelation may not occur, and a precipitate may not form as intended.

In at least one embodiment the concentration of C-ethyl pyrogallol[4]arene dissolved in ethanol is 7.53×10⁻³ M. Upon dispensing 1 mL of this solution into a test solution, as above, the concentration would be 3.77×10⁻⁴ M. The ethanol may be 200 proof ethanol.

In accordance with various embodiments the creation of a pyrogallarene precipitate is the act of chelation and sequestration. Chelation and sequestration may be exhibited by a shift in pyrogallarene absorbance. Purified solid pyrogallarenes are colorless, however solvent or ionic interactions result in the appearance of colors. This may be due to solvatochromism. To the extent pyrogallarenes may visually indicate the chelation of metal ions they may also be referred to in the art as metal ion indicators, complexometric indicators, or metallochromic indicators. Importantly, the presence or absence of various elements, ions, or complexes may be determined by pyrogallarene shifts in absorbance. One example of this is Example A.

### Example A

A stock solution of was made of 400 mg of purified C-propyl pyrogallol[4]arene dissolved in 20 mL of acetone. 1 mL aliquots of this stock solution were dispensed into vials containing 20 mL of deionized water. Upon dispensing this aliquot the aqueous solutions were a colorless white colloid. The colloid appeared to be somewhat translucent. Upon dispensing NaCl into one colorless colloid the color was unchanged. Then various chloride salts of metals were added to colorless C-propyl pyrogallol[4]arene colloids. Upon adding ZnCl₂ the colloid turned light brown. Upon adding MgCl₂ the colloid turned pink. Upon adding Fe(III)Cl₃ the colloid turned grey. Upon adding Cu(I)Cl the colloid turned brown. Upon adding CaCl₂ the colloid turned pink. Upon adding Cr(III)Cl₃ the colloid turned green. Upon adding BaCl₂.2H₂O the colloid turned pink. Upon adding MnCl₂ the colloid turned salmon. Upon adding NiCl₂ the colloid turned salmon.

In Example A, half of the metal salts, ZnCl₂, MgCl₂, CaCl₂, and BaCl_{z}.2H₂O, were colorless in their solid form, before adding them to the colloid. Pyrogallarene chelation is evident as a colorless salts are added to a colorless colloid and varying colors result. Five of the salts were colored in their solid form. However three of the salts, Fe(III)Cl₃, Cu(I)Cl, and NiCl₂, showed a noticeable shift in color upon being added to the colloid. Although Fe(III)Cl₃ was brownish red in its solid form the resulting colloid solution was grey. Cu(I)Cl was green in its solid form but the resulting colloid solution was brown. NiCl₂ was green in its solid form but the resulting colloid solution was pink.

### Example B

where C-ethyl pyrogallarene was saturated in methanol, showed similar results to Example A. One milliliter aliquots of C-ethyl pyrogallarene saturated in methanol were added to 20 mL solutions of deionized 18 MΩ*cm water, creating a colorless and murky colloid. Upon addition of PbCl₂, CaCl₂, and ZnCl₂ a blue precipitate immediately formed and fell out of solution. Some metals required more time for a precipitate to form. 24 hours later a colloid with Cu(I)Cl showed a grey precipitate that fell out of solution. Likewise solutions containing Cr(III)Cl₃, BaCl_{z}.2H₂O, and NiCl_{z} showed a white precipitate 24 hours later.

After air drying all of the water out of a solution containing blue PbCl₂-pyrogallol[4]arene precipitate the precipitate turned colorless. However, upon adding water the colorless precipitate returned to blue, indicating that a solvent, particularly water, may be part of the chelating complex.

### Example C

A stock solution of was made of 100 mg of purified C-ethyl pyrogallol[4]arene dissolved in 20 mL of acetone. 1 mL aliquots of this stock solution were dispensed into vials containing 20 mL of deionized water. Upon dispensing this aliquot the aqueous solutions were colorless white colloids. Various chloride salts were added to the solution including Yttrium(II) chloride hydrate, Tungsten(IV) chloride, Strontium chloride, Titanium chloride, Zirconyl (IV) chloride octahydrate, Aluminum chloride, and Hafnium chloride. All metal salts were colorless except Tungsten(IV) chloride, which was light green, and Titanium chloride, which was a yellow solution. Upon adding Yttrium(II) chloride hydrate a purple precipitate formed. Upon adding Strontium chloride a white precipitate formed. Upon adding Zirconyl (IV) chloride octahydrate a clear grey solution formed. Upon adding Hafnium chloride a grey precipitate began to form. All other colloids appeared unchanged immediately after the addition of the metal. This may be a result of no chelation or slow reaction kinetics for the chelated complex. Finally, one additional salt, colorless Gadolinium Chloride, was tested. Upon its addition to the colorless colloid a purple precipitate formed. As stated previously, various examples may indicate chelation and sequestration by pyrogallarenes on a PPM level, owing to the solubility of various chloride salts. For example, PbCl₂ is very poorly soluble in water.

### Example D

A stock solution of was made of 100 mg of purified C-ethyl pyrogallol[4]arene dissolved in 20 mL of acetone. 1 mL aliquots of this stock solution were dispensed into vials containing 20 mL of water. Sample one contained TDS of 330, Ca of 30 ppm, Mg of 26 ppm, K of 1 ppm, Si of 15 ppm, bicarbonate of 360 ppm, Pb of <0.001, and a pH of 7.2. Sample two contained TDS of 222, Ca of 18 ppm, Mg of 15 ppm, K of 5.3 ppm, Si of 93 ppm, bicarbonate of 152 ppm, and a pH of 7.7. Sample three contained TDS of 44, Ca of 5 ppm, Mg of 1 ppm, K of 0 ppm, bicarbonate of 20 ppm, and a pH of 7.8. Sample four had a pH of 9.4. Upon dispensing 1 mL of this aliquot, sample one quickly produced a dense blue precipitate that fell out of solution, over the period of a few minutes sample two produced a diffuse blue precipitate that gradually fell out of solution, sample three did not produce a precipitate and remained a colorless, white colloid for several days, and sample four readily shifted to a pink colored solution due to the high pH.

Pyrogallarenes, including C-methyl Pyrogallol[4]arene, C-ethyl Pyrogallol[4]arene, and C-propyl pyrogallol[4]arene, have a high affinity for metals, even in a homogeneous solid state reaction. For example, a stainless steel spatula in contact with these freshly-synthesized and not completely dry (nearly dry) pyrogallarenes results in a color shift from white to blue where the spatula came in contact with the pyrogallol[4]arene. This example may illustrate, as in previous examples, that a chelation reaction has occurred.

Upon dispensing solid, colorless C-ethyl pyrogallol[4]arene into an unpurified aqueous solution the C-ethyl pyrogallol[4]arene will shift absorbance to a blue color and remain almost completely undissolved. This example may illustrate that a heterogeneous chelation reaction has occurred, as the C-ethyl pyrogallol[4]arene is mostly insoluble in water. Similar results may be observed by placing solid C-ethyl pyrogallol[4]arene in a purified aqueous solution that has Pb added. These examples resemble the chelation and precipitation reaction that occurs with heterogeneous pyrogallol[4]arenes in an aqueous mixture. In accordance with various embodiments the pyrogallarene selectively precipitates ions out of solution.

Unlike some solids where solubility increases rapidly with increasing temperature the pyrogallarene chelated precipitate is resilient at high temperature. For example, a chelate made from C-propyl pyrogallol[4]arene and PbCl₂ is stable and undissolved at 80° C. A chelate made from C-propyl pyrogallol[4]arene and a variety of metals is also undissolved at 80° C. As an illustrative example, time may have a greater influence on the dissolution of pyrogallarene chelate where the blue precipitate dissolves over the period of a few weeks to create a yellow solution. However, heterogeneous pyrogallol[4]arene chelation complexes appear to dissolve minimally into aqueous solutions, limited by a low Ksp as discussed above.

In at least one embodiment the chromogenic nature of the pyrogallarene chelate is associated with the electronic interactions of the calixarene cavity, a part of the shifting of the HOMO and LUMO of the molecule. In at least one embodiment the chelate color shift is partially due to the supramolecular self-assembly of solvated pyrogallarene units.

The invention encompasses various embodiments. In at least one embodiment chelation may occur with zinc, magnesium, lead, calcium, barium, chromium, manganese, nickle, uranium, or sulfate. In at least one embodiment chelation may occur with Row 4, 5, 6, and 7 metals of the periodic table. In at least one embodiment, chelation may occur with silicon, cobalt, copper, yttrium, zirconium, niobium, lanthanum, cerium, praseodymium, hafnium, or neodymium.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for using a pyrogallarene as a sequestering agent comprising:
dispensing the pyrogallarene into an aqueous solution.

2. The method of claim 1, wherein the pyrogallarene is a pyrogallol[4]arene.

3. The method of claim 2, wherein the pyrogallol[4]arene is C-propyl pyrogallol[4]arene.

4. The method of claim 2, wherein the pyrogallol[4]arene is C-alkyl pyrogallol[4]arene.

5. The method of claim 2, wherein the pyrogallol[4]arene is C-ethyl pyrogallol[4]arene.

6. The method of claim 2, wherein the pyrogallol[4]arene is a homogeneous part of the aqueous solution.

7. The method of claim 2, wherein the pyrogallol[4]arene is a heterogeneous part of the aqueous solution.

8. The method of claim 1, additionally comprising observing a color absorbance shift in a content of the solution.

9. The method of claim 8, wherein the color absorbance shift is an indication of chelation or sequestration.

10. The method of claim 4, additionally comprising dispensing the pyrogallol[4]arene into the aqueous solution after dissolving the pyrogallol[4]arene in an organic solvent.

11. The method of claim 10, wherein the organic solvent comprises one of methanol, ethanol, propanol, butanol, acetone, DMSO, or DMF.

12. The method of claim 11, wherein if the aqueous solution is deionized and pH neutral the pyrogallol[4]arene is suspended as a colorless colloid.

13. The method of claim 11, wherein the pyrogallol[4]arene is concentrated in the organic solvent so as to produce an aqueous colloid.

14. A method for using a pyrogallarene as a homogeneous chelating agent comprising:
exposing the pyrogallarene to metal atoms.

15. The method of claim 14, wherein the pyrogallarenes are exposed to the metal by:
dissolving the pyrogallarane in an organic solvent; and
dispensing the dissolved pyrogallarene into an aqueous solution.

16. The method of claim 14, wherein the pyrogallarene in the aqueous solution has a concentration greater than about 3×10⁻³ M.

17. A method for using one or more solid pyrogallarenes as heterogeneous chelating agents comprising:
exposing the one or more solid pyrogallarenes to metal atoms.

18. The method of claim 17, wherein the one or more solid pyrogallarenes are exposed to metal atoms by dispensing the one or more pyrogallarenes into an aqueous solution containing metal ions.

19. The method of claim 18, further comprising mixing the aqueous solution and/or centrifuging the aqueous solution.
